(19) United States
(12) Patent Application Publication
(10) Pub. No.: US 2014/0309312 A1
(43) Pub. Date: Oct. 16, 2014

(54) SYNTHESIS OF AN IRON BASED MAGNETIC COMPOSITE MATERIAL FOR ANTICANCER DRUG DELIVERY

(71) Applicant: Bo Fan, Ames, IA (US)

(72) Inventor: Bo Fan, Ames, IA (US)

(21) Appl. No.: 14/251,806

(22) Filed: Apr. 14, 2014

Related U.S. Application Data
(60) Provisional application No. 61/811,251, filed on Apr. 12, 2013.

(51) Int. Cl. A61K 47/02

(52) U.S. Cl. CPC A61K 47/02
USPC 514/769

(57) ABSTRACT

A method of synthesizing for an iron based magnetic composite material includes the steps of stirring together potassium hydroxide, iron (II, III) oxide, and water. The method also includes the steps of heating the resulting Fe₃O₄—KOH mixture for a predetermined amount of time. Further, the method includes the step of heating the Fe₃O₄—KOH mixture until consecutive measurements of the Fe₃O₄—KOH mixture are within 10% of each other. Also included in the method if adding calcium hypochlorite and mixing until a magnetic Fe(VI)/Fe₃O4 composite material is formed. Additionally, the method involves preparing and analyzing the magnetic Fe(VI)/Fe₃O4 composite material to determine the amount of ferrate synthesized during from the method.

### CROSS REFERENCE TO RELATED APPLICATIONS

This application claims the benefit of U.S. Provisional Application No. 61/811,251, filed Apr. 12, 2013.

### BACKGROUND OF THE INVENTION

This invention relates to a method of synthesizing an iron composite material. More specifically, the present invention relates to a method of synthesizing an iron based magnetic composite material for anticancer drug delivery.

Ferrate (Fe(VI) or FeO₄²⁻) is considered to be the representative of a new generation of oxidation agents. Given its strong oxidation ability, ferrate can potentially be used for drug delivery, specifically, cancer fighting drug delivery.

Various methods currently exist to synthesize Ferrate. One particular method is using a wet synthesis method which involves the following major reactions:

2FeCl₃+3NaClO+10NaOH->2Na₂FeO₄+9NaCl+5H₂O 2Na₂FeO₄+4KOH->2K₂FeO₄+4NaOH

The combined reaction can be shown as the following:

2FeCl₃+3NaClO+6NaOH+4KOH->2K₂FeO₄+4NaOH+9NaCl+5H₂O

If the above reaction were based on the synthesis of a single mole of ferrate the reaction is as follows:

FeCl₃+1.5NaClO+3NaOH+2KOH->K₂FeO₄+4.5NaCl+2.5H₂O

As shown in the above reactions, this method consumes 1 mole of iron (III) chloride (FeCl₃), 1.5 moles of sodium chloride (NaCl), 3 moles of sodium hydroxide (NaOH), and 2 moles of potassium hydroxide (KOH) to produce a single mole of ferrate, while producing 4.5 moles of byproduct in waste solution—not including water as a byproduct.

Alternatively, a second known method of synthesizing ferrate exists using an electrochemical mechanism. When potassium hydroxide is used as an electrolyte, the cathode and anode reactions are as follows:

6H₂O->3H₂+6OH⁻+6e 2Fe+4KOH+6OH⁻->2K₂FeO₄+3H₂+2H₂O+6e

The overall reaction is:

2Fe+4KOH+4H₂O->2K₂FeO₄6H₂

Using this method appears to be cleaner as the only byproduct is gaseous hydrogen. However, the separation efficiency of K₂FeO₄ is very low and as such, a substantial amount of waste solution is generated using this method. Further, the costs associated with this type of method are prohibitively high.

A third method of synthesizing ferrate is based on reaction among solids. One particular method is as follows:

FeSO₄+2NaClO+4KOH->2K₂FeO₄+2NaCl+K₂SO₄+2H₂O

This method has the advantage of being a short and simple method to synthesize ferrate. Similar to the wet and electrochemical methods, however, a significant amount of waste is involved. Specifically, to generate 1 mole of ferrate consumes 2 moles of sodium hypochlorite (NaClO) while producing 3 moles of byproduct, which increases the cost of this method of synthesis and harms the environment.

As noted, despite these advances in the art, problems remain. In particular, problems exist with synthesizing ferrate in a cost effective way. Further, problems exist with a method of synthesizing ferrate that is environmentally friendly. Still further, problems exist with synthesizing ferrate in such a way that there is little byproduct or waste.

Thus it is the primary object of the invention to provide a method of synthesizing ferrate that improves upon the state of the art.

Another object of the invention is to provide a method of synthesizing ferrate that is both cost effective and environmentally friendly.

Yet another object of the invention is to provide a method of synthesizing ferrate that has minimal waste or byproduct.

Another object of the invention is to provide a method of synthesizing ferrate that is simple.

Yet another object of the invention is to provide a method of synthesizing ferrate that is short.

Another object of the invention is to provide a method of synthesizing a partially oxidated composite material.

Yet another object of the invention is to provide a method of synthesizing an iron based composite material for anticancer drug deliver.

These and other objects, features, or advantages of the present invention will become apparent from the specification and claims.

### SUMMARY OF THE INVENTION

A method of synthesizing for an iron based magnetic composite material includes the steps of stirring together potassium hydroxide, iron (II, III) oxide, and water. The method also includes the steps of heating the resulting Fe₃O₄—KOH mixture for a predetermined amount of time. Further, the method includes the step of heating the Fe₃O₄—KOH mixture until consecutive measurements of the Fe₃O₄—KOH mixture are within 10% of each other. Also included in the method if adding calcium hypochlorite and mixing until a magnetic Fe(VI)/Fe₃O4 composite material is formed. Additionally, the method involves preparing and analyzing the magnetic Fe(VI)/Fe₃O4 composite material to determine the amount of ferrate synthesized during from the method.

### BRIEF DESCRIPTION OF DRAWINGS [INCOMPLETE]

FIG. 1 is a perspective view of the system to synthesize an iron based magnetic composite material;

FIG. 2 is a perspective view of the system to synthesize an iron based magnetic composite material;

FIG. 3 is a perspective view of the system to synthesize an iron based magnetic composite material;

FIG. 4 is a perspective view of the system to synthesize an iron based magnetic composite material; and

FIG. 5 is a perspective view of the system to synthesize an iron based magnetic composite material.

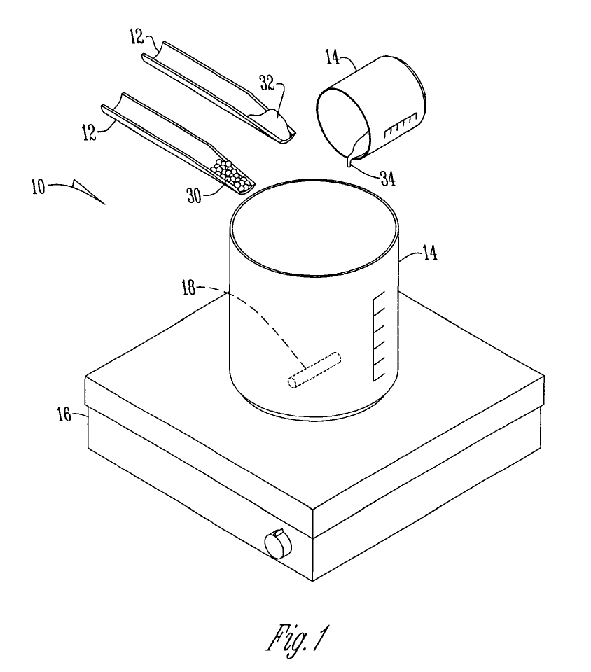

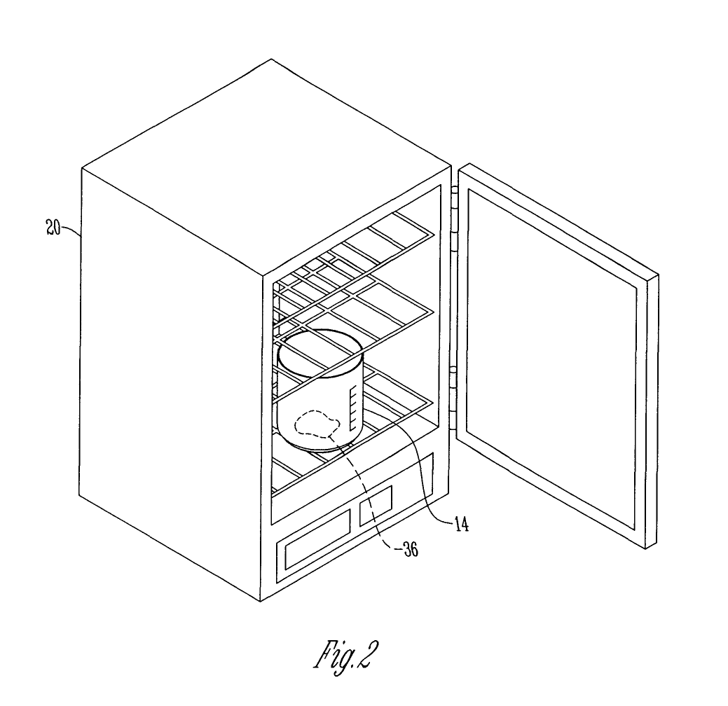

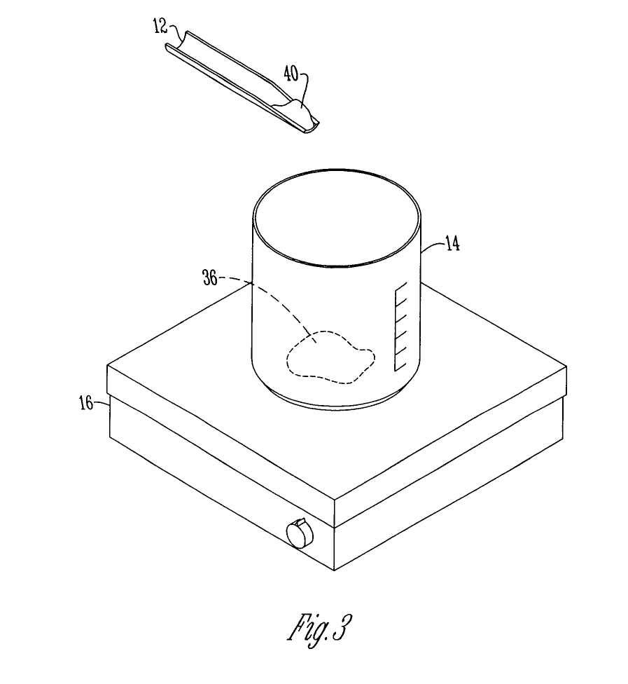

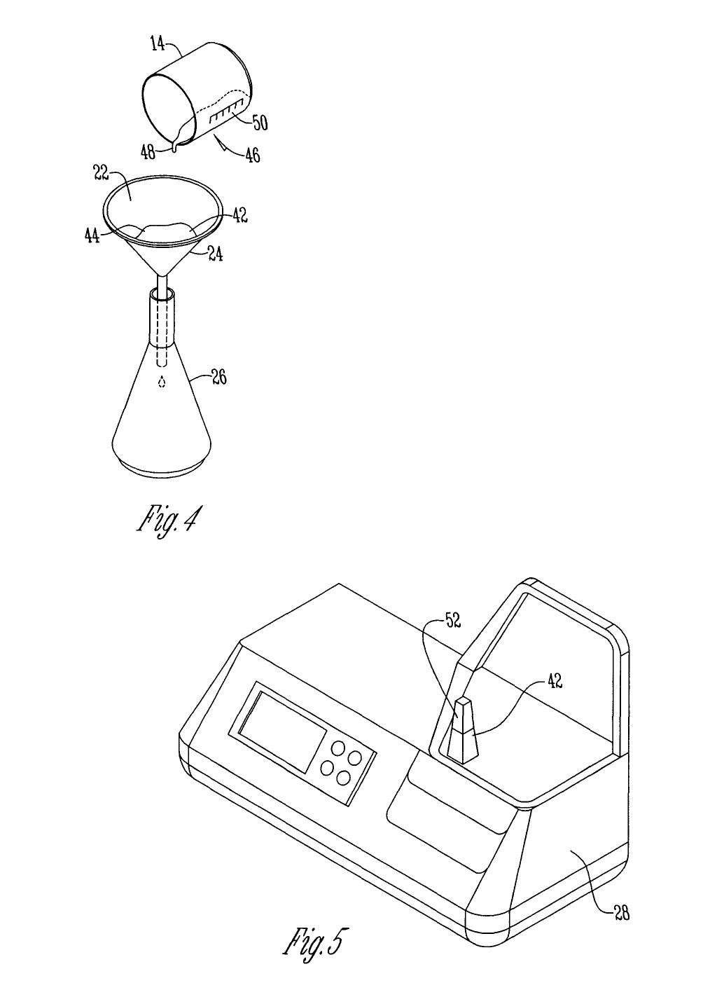

### DESCRIPTION OF THE PREFERRED EMBODIMENT

The figures show a system 10 utilized to synthesize an iron based magnetic composite material. The system 10 includes a plurality of scoops 12, a plurality of beakers 14, a stir plate 16, a stir bar 18, an oven 20, a filter paper 22, a funnel 24, a flask 26, and a UV/vis spectrometer 28.

The method begins with using a scoop 12 to obtain a predetermined amount of potassium hydroxide 30, preferably 1 mole of potassium hydroxide 30. The potassium hydroxide 30 loaded onto iron (II, III) oxide 32 (Fe₃O₄) in a beaker 14, preferably 3 moles of iron (II, III) oxide 32 is used. In other embodiments the amount of potassium hydroxide 30 and iron (II, III) oxide 32 are added in amounts in an approximate 1:3 stoichiometric ratio. Then water 34 is added to the beaker 14, preferably 150 mL of water 34 is added.

The beaker 14 is then placed on the stirred for a predetermined amount of time to form a Fe₃O₄—KOH mixture 36, preferably for 30 minutes. In one embodiment of the method the beaker 14 is placed on a stir plate 16 and stirred using a stir bar 18.

After stirring, the beaker 16 holding the Fe₃O₄—KOH mixture 36 is placed in the oven 20 for a predetermined amount of time at a predetermined temperature. In one embodiment the amount of time is 30 minutes and the predetermined temperature is 85° C. In another embodiment the Fe₃O₄—KOH mixture 36 is heated until the difference of two consecutive measurements of the weight of the Fe₃O₄—KOH mixture 34 is less than 10%.

After removing the beaker 14 from the oven 20 a predetermined amount of calcium hypochlorite 40 (Ca(ClO)₂) is added to the beaker 14. Preferably 7.5 moles of calcium hypochlorite 40 is added. The contents of the beaker 14 is then mixed for a predetermined amount of time to form a magnetic Fe(VI)/Fe3O4 composite material 42. In one embodiment the amount of time is 10 hours. In another embodiment the contents of the beaker 14 is stirred using the stir plate 16 and the stir bar 18.

In order to test the amount of ferrate 44 synthesized from the iron (II, III) oxide 32 the magnetic Fe(VI)/Fe₃O4 composite material 42 is washed. In one embodiment the magnetic Fe(VI)/Fe3O4 composite material 42 is washed using a buffer solution 46, preferably with a pH of about 9.0. In another embodiment the buffer solution 46 is prepared using Na₂B₄O₇ 48, preferably 2.2 mM, and Na₂HPO₄ 50, preferably 2.2 mM.

The magnetic Fe(VI)/Fe₃O4 composite material 42 is then filtered. In one embodiment the Magnetic Fe(VI)/Fe3O4 composite material 42 is filtered using the flask 26 and the funnel 24 with funnel paper 22. Other methods of filtering, such as vacuum filtration are also contemplated.

Then a sample 52 is prepared from the magnetic Fe(VI)/Fe₃O4 composite material 42. The sample 52 is then analyzed in the UV/vis spectrometer 28. In one embodiment the sample 52 is analyzed at 510 nm, which is the characteristic wavelength of ferrate 44. In addition to UV/vis, other methods of analyzing the ferrate 44 conversion are contemplated.

Further, the magnetic Fe(VI)/Fe₃O4 composite material 42 can be prepared by partial oxidation. The magnetic property of the magnetic Fe(VI)/Fe₃O4 composite material 42 resulting from the unoxidized iron (II, III) oxide can be used to deliver anticancer drugs, and in particular cancer fighting drugs.

Additionally, as seen in the following reaction the method reduces cost and waste:

6Fe₃O₄+15Ca(ClO)₂+36KOH->18K₂FeO₄+15CaCl₂+18H₂O

The above reaction shows for every mole of ferrate 44 that is produced only 0.833 moles of calcium hypochlorite 40 is consumed compared to 1.5 and 2 moles in the previously noted methods. Additionally, only 2 moles of potassium hydroxide 30 are consumed compared to 25 moles of sodium hydroxide and 4 moles of potassium hydroxide 30 as indicated in the above reactions.

Furthermore, this method of synthesis generates less byproduct since there is only 0.83 moles of calcium chloride (CaCl₂) is produced compared to 4.5 moles of sodium chloride and 3 moles of byproduct as seen in the other methods of synthesizing ferrate 44. Thus, at least all of the stated problems have been overcome

What is claimed is:

1. A method of synthesizing an iron based magnetic composite material comprising the steps of:
loading potassium hydroxide onto iron (II, III) oxide;
adding water;
stirring the potassium hydroxide, iron (II, III) oxide, and water to form a Fe₃O₄—KOH mixture;
heating the Fe₃O₄—KOH mixture;
adding calcium hypochlorite; and
mixing the Fe₃O₄—KOH mixture and the calcium hypochlorite to form a magnetic Fe(VI)/Fe₃O4 composite material.

2. The method of claim 1 wherein the amount of the potassium hydroxide loaded is in about a 1:3 stoichiometric ratio to the amount of iron (II, III) oxide.

3. The method of claim 1 wherein the amount of water added is 150 mL.

4. The method of claim 1 wherein the potassium hydroxide, iron (II, III) oxide, and water are stirred for thirty minutes.

5. The method of claim 1 wherein the Fe₃O₄—KOH mixture is heated using an oven set to 85° C.

6. The method of claim 1 wherein the Fe₃O₄—KOH mixture is heated until the difference in weight is less than 10% in two consecutive measurements.

7. The method of claim 1 wherein the amount of calcium hypochlorite is 7.5 moles.

8. The method of claim 1 wherein the Fe₃O₄—KOH mixture and calcium hypochlorite are mixed for ten hours.

9. The method of claim 1 wherein the magnetic Fe(VI)/Fe₃O4 composite material is prepared with partial oxidation.

10. The method of claim 9 wherein the magnetic Fe(VI)/Fe₃O4 composite material is used for drug delivery.

11. The method of claim 1 further comprising the steps of washing the magnetic Fe(VI)/Fe₃O4 composite material with a buffer solution; filtering the magnetic Fe(VI)/Fe₃O4 composite material; and analyzing the sample to determine an amount of ferrate converted.

12. The method of claim 11 wherein the sample is analyzed at 510 nm in a UV/via spectrometer

13. The method of claim 11 wherein the buffer solution has a pH of about 9.0.

14. The method of claim 11 wherein the buffer solution is prepared using Na₂B₄O₇ and Na₂HPO₄.

15. The method of claim 14 wherein the amount of Na₂B₄O₇ used is 2.2 mM.

16. The method of claim 14 wherein the amount of Na₂HPO₄ used is 2.2 mM.
